SEQUENCE LISTING (24 sequences)

SEQ ID NO: 1 - protein, 1676 aa, Homo sapiens
MGLLGILCFLIFLGKTWGQEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVTLNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEILRPRRTLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGVGISNTGICVADTVKAKVFKDVFLEMNIPYSVVRGEQIQLKGTVYNYRTSGMQFCVKMSAVEGICTSESPVIDHQGTKSSKCVRQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

SEQ ID NO: 2 - protein, 1676 aa, Macaca fascicularis
MGLLGILCFLIFLGKTWGQEQTYVISAPKIFRVGASENIVIQVYGYTEAFDATISIKSYPDKKFSYSSGHVHLSSENKFQNSAVLTIQPKQLPGGQNQVSYVYLEVVSKHFSKSKKIPITYDNGFLFIHTDKPVYTPDQSVKVRVYSLNDDLKPAKRETVLTFIDPEGSEIDMVEEIDHIGIISFPDFKIPSNPRYGMWTIQAKYKEDFSTTGTAFFEVKEYVLPHFSVSVEPESNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYSIKVQVKDALDQLVGGVPVTLNAQTIDVNQETSDLEPRKSVTRVDDGVASFVVNLPSGVTVLEFNVKTDAPDLPDENQAREGYRAIAYSSLSQSYLYIDWTDNHKALLVGEYLNIIVTPKSPYIDKITHYNYLILSKGKIIHFGTREKLSDASYQSINIPVTQNMVPSSRLLVYYIVTGEQTAELVSDSVWLNIEEKCGNQLQVHLSPDADTYSPGQTVSLNMVTGMDSWVALTAVDSAVYGVQRRAKKPLERVFQFLEKSDLGCGAGGGLNNANVFHLAGLTFLTNANADDSQENDEPCKEIIRPRRMLQEKIEEIAAKYKHLVVKKCCYDGVRINHDETCEQRAARISVGPRCVKAFTECCVVASQLRANNSHKDLQLGRLHMKTLLPVSKPEIRSYFPESWLWEVHLVPRRKQLQFALPDSVTTWEIQGVGISNSGICVADTIKAKVFKDVFLEMNIPYSVVRGEQVQLKGTVYNYRTSGMQFCVKMSAVEGICTSESPVIDHQGTKSSKCVRQKVEGSSNHLVTFTVLPLEIGLQNINFSLETSFGKEILVKSLRVVPEGVKRESYSGITLDPRGIYGTISRRKEFPYRIPLDLVPKTEIKRILSVKGLLVGEILSAVLSREGINILTHLPKGSAEAELMSVVPVFYVFHYLETGNHWNIFHSDPLIEKRNLEKKLKEGMVSIMSYRNADYSYSVWKGGSASTWLTAFALRVLGQVHKYVEQNQNSICNSLLWLVENYQLDNGSFKENSQYQPIKLQGTLPVEARENSLYLTAFTVIGIRKAFDICPLVKINTALIKADTFLLENTLPAQSTFTLAISAYALSLGDKTHPQFRSIVSALKREALVKGNPPIYRFWKDSLQHKDSSVPNTGTARMVETTAYALLTSLNLKDINYVNPIIKWLSEEQRYGGGFYSTQDTINAIEGLTEYSLLVKQLRLNMDIDVAYKHKGPLHNYKMTDKNFLGRPVEVLLNDDLVVSTGFGSGLATVHVTTVVHKTSTSEEVCSFYLKIDTQDIEASHYRGYGNSDYKRIVACASYKPSKEESSSGSSHAVMDISLPTGINANEEDLKALVEGVDQLFTDYQIKDGHVILQLNSIPSSDFLCVRFRIFELFEVGFLSPATFTVYEYHRPDKQCTMFYSTSNIKIQKVCEGATCKCIEADCGQMQKELDLTISAETRKQTACNPEIAYAYKVIITSITTENVFVKYKATLLDIYKTGEAVAEKDSEITFIKKVTCTNAELVKGRQYLIMGKEALQIKYNFTFRYIYPLDSLTWIEYWPRDTTCSSCQAFLANLDEFAEDIFLNGC

SEQ ID NO: 3 - protein, 1676 aa, Homo sapiens
MGLLGILCFLIFLGKTWGQEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVTLNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEILRPRRTLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGVGISNTGICVADTVKAKVFKDVFLEMNIPYSVVRGEQIQLKGTVYNYRTSGMQFCVKMSAVEGICTSESPVIDHQGTKSSKCVHQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

SEQ ID NO: 4 - protein, 1676 aa, Homo sapiens
MGLLGILCFLIFLGKTWGQEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVTLNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEILRPRRTLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGVGISNTGICVADTVKAKVFKDVFLEMNIPYSVVRGEQIQLKGTVYNYRTSGMQFCVKMSAVEGICTSESPVIDHQGTKSSKCVCQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

SEQ ID NO: 5 - protein, 5 aa, Homo sapiens
SYAIS

SEQ ID NO: 6 - protein, 17 aa, Homo sapiens
GIGPFFGTANYAQKFQG

SEQ ID NO: 7 - protein, 7 aa, Homo sapiens
DTPYFDY

SEQ ID NO: 8 - protein, 11 aa, Homo sapiens
SGDSIPNYYVY

SEQ ID NO: 9 - protein, 7 aa, Homo sapiens
DDSNRPS

SEQ ID NO: 10 - protein, 11 aa, Homo sapiens
QSFDSSLNAEV

SEQ ID NO: 11 - protein, 116 aa, Homo sapiens
EVQLVQSGAEVKKPGSSVKVSCKASGGTFSSYAISWVRQAPGQGLEWMGGIGPFFGTANYAQKFQGRVTITADESTSTAYMELSSLRSEDTAVYYCARDTPYFDYWGQGTLVTVSS

SEQ ID NO: 12 - protein, 108 aa, Homo sapiens
SYELTQPLSVSVALGQTARITCSGDSIPNYYVYWYQQKPGQAPVLVIYDDSNRPSGIPERFSGSNSGNTATLTISRAQAGDEADYYCQSFDSSLNAEVFGGGTKLTVL

SEQ ID NO: 13 - protein, 446 aa, Homo sapiens
EVQLVQSGAEVKKPGSSVKVSCKASGGTFSSYAISWVRQAPGQGLEWMGGIGPFFGTANYAQKFQGRVTITADESTSTAYMELSSLRSEDTAVYYCARDTPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 214 aa, Homo sapiens
SYELTQPLSVSVALGQTARITCSGDSIPNYYVYWYQQKPGQAPVLVIYDDSNRPSGIPERFSGSNSGNTATLTISRAQAGDEADYYCQSFDSSLNAEVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 15 - protein, 5 aa, Artificial
  eculizumab HCDR1
NYWIQ

SEQ ID NO: 16 - protein, 17 aa, Artificial
  eculizumab HCDR2
EILPGSGSTEYTENFKD

SEQ ID NO: 17 - protein, 13 aa, Artificial
  eculizumab HCDR3
YFFGSSPNWYFDV

SEQ ID NO: 18 - protein, 11 aa, Artificial
  eculizumab LCDR1
GASENIYGALN

SEQ ID NO: 19 - protein, 7 aa, Artificial
  eculizumab LCDR2
GATNLAD

SEQ ID NO: 20 - protein, 9 aa, Artificial
  eculizumab LCDR3
QNVLNTPLT

SEQ ID NO: 21 - protein, 123 aa, Artificial
  eculizumab VH
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSA

SEQ ID NO: 22 - protein, 131 aa, Artificial
  eculizumab VL
MDMRVPAQLLGLLLLWLRGARCDIQMTQSPSSLSASVGDRVTITCGASENIYGALNWYQQKPGKAPKLLIYGATNLADGVPSRFSGSGSGTDFTLTISSLQPEDFATYYCQNVLNTPLTFGQGTKVEIKRT

SEQ ID NO: 23 - protein, 448 aa, Artificial
  eculizumab HC
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 236 aa, Artificial
  eculizumab LC
MDMRVPAQLLGLLLLWLRGARCDIQMTQSPSSLSASVGDRVTITCGASENIYGALNWYQQKPGKAPKLLIYGATNLADGVPSRFSGSGSGTDFTLTISSLQPEDFATYYCQNVLNTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2019/0225678 A1
(43) Pub. Date: Jul. 25, 2019

(54) ANTI-C5 ANTIBODY FOR TREATING PATIENTS WITH COMPLEMENT C5 POLYMORPHISM

(71) Applicant: Novartis AG, Basel (CH)

(72) Inventors: Florian Muellershausen, Basel (CH); Mark Milton, Belmont (CH); Leslie Ngozi Anuna Johnson, Jamaica Plain, MA (US)

(21) Appl. No.: 16/306,654

(22) PCT Filed: Jun. 1, 2017
(86) PCT No.: PCT/IB2017/053245

§ 371 Date: Dec. 3, 2018

Related U.S. Application Data
(60) Provisional application No. 62/346,683, filed on Jun. 7, 2016.

(51) Int. Cl. C07K 16/18; A61P 7/04; C12Q 1/6827

(52) U.S. Cl. CPC C07K 16/18; C12Q 1/6827; A61P 7/04

(57) ABSTRACT

The present invention relates generally to an anti-C5 antibody or antigen binding fragment thereof for use in the prophylaxis or treatment of a complement related disease or disorder in a patient having a polymorphism in complement C5 protein.

### FIELD OF THE INVENTION

The present invention relates to an anti-C5 antibody or antigen binding fragment thereof for use in the prevention or treatment of a complement related disease or disorder in a patient having a polymorphism or mutation within the complement C5 protein.

### BACKGROUND OF THE INVENTION

Complement, a principal component of the innate immune system, is important in host defense. Complement acts to protect against infections, to link adaptive and innate immunity, and to dispose of immune complexes and the products of inflammatory injury (Walport 2001). The complement system consists of over 25 plasma proteins that work through three known activation pathways: classical (antibody complexes), lectin (lectin complexes) and alternative (spontaneous hydrolysis of the soluble complement protein C3).

The complement component C5 is an approximately 189 kDa protein (without considering possible glycosylation) synthesized primarily in the liver as a single-chain precursor molecule. C5 has been shown to also be synthesized by macrophages and specific types of epithelial cells and fibroblasts but the relative contribution of the different tissues to the serum concentrations of C5 is unknown. All three complement pathways converge at C3 activation. The major activation product of C3, C3b, is an essential component of C5 convertases. It has been proposed that molecules of C3b associate with the C3 convertases to form C5 convertases when levels of complement activation are high. This association modulates the activity of the enzyme, causing it to preferentially cleave complement component C5 instead of C3 (M. Jore et al., Nature Structural & Molecular Biology, 2016 Nature America). The C5alpha chain is cleaved by C5 convertases, which are formed during the complement activation process, to form C5a and C5a′ chain C5a′ chain and C513 chain together form C5b.

Human C5 (Uniprot entry P01031) is a secreted, multi-domain glycoprotein consisting of an α-chain (999 amino-acids) and a β-chain (655 amino-acids) linked by a disulfide bridge. The peptide bond between Arg751 and Leu752 within the α-chain is cleaved by C5 convertases to generate the small, 74 amino-acid long C5a fragment and the large C5b fragment (1580 amino-acids). The conversion of C5 to C5b involves large conformational changes and leads to subsequent C6 binding.

Human C5 has been crystallized (Discipio et al 1998; Acta Crystallogr Sect D: Biol Crystallogr; 54:643-646). Determination of the three-dimensional structure of the C5 protein by protein crystallography at 3.1 Å resolution has shown that C5 is a multi-domain protein: C5 contains eight MG domains (MG1-MG8), the CUB domain, the C5d domain, the C5a domain (also called ‘anaphylatoxin’) and an extended linker region packed between MG1-MG2 and MG4-MG6 (Fredslund et al; Nat Immunol; 9:753-760, 2008).

C5a is a major anaphylatoxin involved in chemotaxis of neutrophils, endothelial cell activation and release of pro-inflammatory cytokines. These functions of C5a require binding to its receptor, C5aR. C5b sequentially recruits C6, C7, C8 and C9 in a non-enzymatic manner to form the membrane attack complex (MAC). MAC forms a lytic pore in the target membrane and kills the pathogen. While the functions of C5a and C5b aid in killing the pathogen, they can also be responsible for generating an excess inflammatory response, which can damage host cells. Therefore, C5 functions are tightly regulated by interaction with other proteins in the host. The regulatory proteins can either be host generated or pathogenic factors.

Dysregulated complement activation can result in disease phenotypes that can be collectively referred to as complement related diseases or disorders. For example they can be triggered by dysregulated C3 and/or C5 activation, in particular by excessive C5a- and/or MAC-dependent activities. Complement C5-related diseases or disorders, where there is a significant C5-complement dysregulation component are specific complement related diseases or disorders.

An example of a C5 complement related disease is Paroxysmal Nocturnal Hemoglobinuria (PNH). PNH is a life-threatening disease with high morbidity that affects the blood wherein red blood cells are compromised and then destroyed more rapidly than normal red blood cells. Current PNH treatments involve C5 blockade, which results in the preservation of the critical immune-protective and immune-regulatory functions of upstream components that culminate in C3b-mediated opsonization and immune clearance. Eculizumab (Soliris®, Alexion Pharmaceuticals), a humanized monoclonal antibody that specifically binds to the terminal complement protein C5 inhibiting its cleavage into C5a and C5b by C5 convertases, is shown to be effective in treatment of PNH, and is the only drug approved for PNH.

Eculizumab is also approved for atypical Hemolytic Uremic Syndrome (aHUS). aHUS is an extremely rare, life-threatening, progressive disease that frequently has a genetic component. In most cases it is caused by chronic, uncontrolled activation of the complement system.

In Japan, of 345 patients with PNH who received eculizumab, 11 patients had a poor response. All 11 of these Japanese patients had a single missense C5 heterozygous mutation, c.2654G→A, which predicts the polymorphism p.Arg885His. The prevalence of this mutation among the patients with PNH (3.2%) was similar to that among healthy Japanese people (3.5%). This polymorphism was also identified in a Han Chinese population. In addition, a patient in Argentina of Asian ancestry who had a poor response to eculizumab had a different mutation, c.2653C→T, which predicts the polymorphism p.Arg885Cys. Non-mutant and mutant C5 both caused hemolysis in vitro, but only non-mutant C5 bound to and was blocked by eculizumab. The functional capacity of C5 variants with mutations at Arg885, together with their failure to undergo blockade by eculizumab, account for the poor response to this agent in patients who carry these mutations (Nishimura et al., New Engl J Med 2014; 370; 7). Due to a lack of an alternative to eculizumab treatment, patients who are not responsive to eculizumab treatment cannot be treated. Thus, despite current treatment options for treating diseases and disorders associated with the classical and/or alternative component pathways, particularly PNH, there is a need for finding treatments suitable for non-responding patient populations.

### SUMMARY OF THE INVENTION

The present invention relates to an anti-C5 antibody or antigen binding fragment thereof for use in the prophylaxis or treatment of a complement related disease or disorder, such as a C5-complement related disease or disorder, e.g. PNH or aHUS, in a patient who has a mutation or polymorphism within the eculizumab epitope of the complement C5 protein.

Various (enumerated) embodiments of the disclosure are described herein. It will be recognized that features specified in each embodiment may be combined with other specified features to provide further embodiments of the present disclosure. There is provided an anti-C5 antibody or antigen binding fragment, e.g. an anti-C5 antibody or antigen binding fragment which binds to an epitope of the C5 protein that is distinct and optionally remote from the eculizumab epitope, e.g. tesidolumab or an antigen binding fragment thereof, for use as a medicament in a method comprising administering an effective amount of an anti-C5 antibody capable of inhibiting the complement activation in a patient who has a mutation or polymorphism within the eculizumab epitope of the complement C5 protein, e.g. a p.Arg885 polymorphism, to said patient.

There is provided an anti-C5 antibody or antigen binding fragment for use in a method of treating a patient who has a mutation or polymorphism within the eculizumab epitope of the complement C5 protein, e.g. a p.Arg885 polymorphism in complement C5 protein, wherein the method comprises administering an effective amount of an anti-C5 antibody to said patient, and wherein said anti-C5 antibody is capable of inhibiting the complement activation in said patient.

There is provided an anti-C5 antibody or antigen binding fragment, e.g. tesidolumab or antigen binding fragment thereof, for use as a medicament in a method of treating a patient who has a mutation or polymorphism within the eculizumab epitope of the complement C5 protein, e.g. a p.Arg885 polymorphism, wherein said method comprises the step of determining from a biological sample obtained from a patient whether the C5 complement protein of the patient comprises a mutation or a polymorphism within the eculizumab epitope, wherein the biological sample is of tissue or fluid isolated from said patient.

There is provided an anti-C5 antibody or antigen binding fragment, e.g. tesidolumab or antigen binding fragment thereof, for use in a method of treating a complement related disease or disorder in a patient in need thereof, the method comprising:
a. taking a biological sample from the patient
b. screening for mutations or polymorphisms in the gene encoding C5 of said patient
c. determining whether the patient has a mutation or polymorphism within the eculizumab epitope of the complement C5 protein, e.g. the p.Arg885 polymorphism in the C5 complement protein,
d. administering an effective amount of an anti-C5 antibody capable of inhibiting the C5 complement activation in a patient who has such a mutation or polymorphism to the patient having said mutation or polymorphism, wherein the biological sample is of tissue or fluid isolated from the patient.

There is furthermore provided an anti-C5 antibody or antigen binding fragment capable of binding to the C5 complement protein outside of the eculizumab epitope, e.g. tesidolumab or antigen binding fragment thereof, for use in a method of treating a complement related disease or disorder, e.g. PNH or aHUS, the method comprising:
a. determining from a biological sample obtained from a patient whether the patient has a mutation or polymorphism within the eculizumab epitope of the complement C5 protein, e.g. the p.Arg885 polymorphism in the C5 complement protein, wherein the biological sample is of tissue or fluid isolated from the patient; and
b. administering an effective amount of said anti-C5 antibody or antigen binding fragment, e.g. tesidolumab or antigen binding fragment thereof, to said patient.

There is also provided an anti-C5 antibody or antigen binding fragment, e.g. an anti-C5 antibody or antigen binding fragment that binds to a C5 protein epitope that is distinct and optionally remote from the eculizumab epitope, e.g. tesidolumab or antigen binding fragment thereof, for use in the prevention or treatment of a complement related disease or disorder in a patient in need thereof wherein the patient does not respond to eculizumab treatment.

There is also provided an anti-C5 antibody or antigen binding fragment that binds to a C5 protein epitope that is distinct and optionally remote from the eculizumab epitope for use in the prophylaxis or treatment of PNH or aHUS; and specific dosing regimens for such uses.

Furthermore there is provided tesidolumab, or an antigen binding fragment thereof, for use in the prophylaxis or treatment of PNH or aHUS; and specific dosing regimens for such uses.

### BRIEF DESCRIPTION OF THE DRAWINGS

FIG. 1 Close-up view of the tesidolumab-C5 interface. The CUB and TED/C5d domains of C5 (grey cartoon) are in dark and light grey, respectively, with peptide stretches contributing to the epitope visible in a dashed line box. The tesidolumab Fab is also indicated.

FIG. 2 C5 polymorphism at position 885 does not affect the epitope recognized by tesidolumab. Overall view of the C5 (grey cartoon) complex with the tesidolumab Fab (black cartoon) showing that the location of Arg885 is on the opposite side of C5 with respect to the epitope of tesidolumab.

FIG. 3 Membrane Attack Complex (MAC) formation C5 (wt or mutant) spiked into C5-depleted serum. Tesidolumab but not eculizumab inhibits the activity of mutant C5.

FIG. 4 Tesidolumab shows anti-hemolytic effects in C5 variant and non-variant PNH.

FIG. 5 Comparison of anti-hemolytic effects in C5 variant and non-variant PNH of tesidolumab and eculizumab.

### DETAILED DESCRIPTION OF THE INVENTION

Currently, the most effective treatment available for PNH is the anti-C5 antibody eculizumab. Recently, it has been discovered that a certain patient subpopulation with mutations at Arg885 in the complement C5 protein, respond poorly to treatment with eculizumab. The inventors have identified an anti-C5 antibody or antigen binding fragment thereof, which recognizes the C5 variants with mutations at Arg885, and which is suitable for use in the treatment of a C5 complement related disease or disorder in a patient who has a p.Arg885 polymorphism in complement C5 protein.

In one aspect the present invention relates to an anti-C5 antibody or antigen binding fragment thereof for use in the prevention or treatment of a C5 complement related disease or disorder in a patient who has a p.Arg885 polymorphism in complement C5 protein.

The terms “complement C5 protein” or “C5” or “C5 protein” or “C5 complement protein” are used interchangeably, and also refer to the complement C5 protein in different species. For example, human C5 has the sequence as set in SEQ ID NO: 1 in Table 1 and cynomolgus C5 has the sequence as set in SEQ ID NO: 2 in Table 1 (Macaca fascicularis). Human C5 can be obtained from Quidel (Cat. Number A403). Human C5 (Uniprot entry P01031) is a secreted, multi-domain glycoprotein consisting of an α-chain (999 amino-acids) and a β-chain (655 amino-acids) linked by a disulfide bridge. The peptide bond between Arg751 and Leu752 of the α-chain is cleaved by C5 convertases to generate the small, 74 amino-acid long C5a fragment and the large C5b fragment (1580 amino-acids). The conversion of C5 to C5b involves large conformational changes and leads to subsequent C6 binding.

Two genetic variants of human C5 at position 885, the Arg885 to His and Arg885 to Cys variants, have been discovered. A single missense C5 heterozygous mutation, c.2654G→A, which predicts the polymorphism p.Arg885His, has been described in Japanese and Han Chinese populations (SEQ ID NO: 3 of Table 1).

Another mutation, c.2653C→T, which predicts p.Arg885Cys, was described in an Argentinian population of Asian ancestry (SEQ ID NO: 4 of Table 1). Only non-mutant C5 bound to and was blocked by eculizumab. These two genetic variants of human C5 at position 885 have been observed in PNH patients showing a poor response to eculizumab treatment (Nishimura et al., New Engl J Med 2014; 370; 7). These C5 variants were functional but not blocked by eculizumab. Arg885 is found within the MG7 domain of C5, and is positioned in (or near) the eculizumab epitope.

Thus, in one embodiment, the present invention relates to an anti-C5 antibody or antigen binding fragment thereof for use in the prevention or treatment of a complement related disease or disorder, e.g. a C5 complement related disease or disorder, in a patient who has a mutation or polymorphism within the MG7 domain of complement C5 protein or within the eculizumab epitope of complement C5 protein, e.g. a p.Arg885 polymorphism in complement C5 protein, wherein said mutation or polymorphism is a p.Arg885 polymorphism. In another embodiment, the present invention relates to an anti-C5 antibody or antigen binding fragment thereof for use in the prophylaxis or treatment of a complement related disease or disorder, e.g. a C5 complement related disease or disorder, in a patient who has a p.Arg885 polymorphism in complement C5 protein, wherein said p.Arg885 is a p.Arg885Cys polymorphism or a p.Arg885His polymorphism.

The term “polymorphism”, as used herein, refers to DNA sequence variations that occur when a nucleotide in the genome sequence is altered. Single nucleotide polymorphisms (SNPs) are DNA sequence variations that occur when a single nucleotide in the genome sequence is altered. The term “a p.Arg885 polymorphism in complement C5 protein”, as used herein, refers to a missense C5 heterozygous mutation leading to substitution of Arg885 in C5 by another amino acid, e.g. His in p.Arg885His, Cys in p.Arg885Cys.

C5 polymorphism can be detected by assaying a sample obtained from a patient. The term “assaying” is used to refer to the act of identifying, screening, probing or determining, which act may be performed by any conventional means. For example, a sample may be assayed for the presence of a particular marker by using an ELISA assay, a Northern blot, imaging, etc. to detect whether that marker is present in the sample. The terms “assaying” and “determining” contemplate a transformation of matter, e.g., a transformation of a biological sample, e.g., a blood sample or other tissue sample, from one state to another by means of subjecting that sample to physical testing. Further, as used herein, the terms “assaying” and “determining” are used to mean testing and/or measuring. The phrase “assaying a biological sample from the patient for . . . ” and the like is used to mean that a sample may be tested (either directly or indirectly) for either the presence or absence of a given factor or for the level of a particular factor. It will be understood that, in a situation where the presence of a substance denotes one probability and the absence of a substance denotes a different probability, then either the presence or the absence of such substance may be used to guide a therapeutic decision.

The step of assaying comprises a technique selected from the group consisting of Northern blot analysis, polymerase chain reaction (PCR), reverse transcription-polymerase chain reaction (RT-PCR), TaqMan-based assays, direct sequencing, dynamic allele-specific hybridization, high-density oligonucleotide SNP arrays, restriction fragment length polymorphism (RFLP) assays, primer extension assays, oligonucleotide ligase assays, analysis of single strand conformation polymorphism, temperature gradient gel electrophoresis (TGGE), denaturing high performance liquid chromatography, high-resolution melting analysis, DNA mismatch-binding protein assays, SNPLex®, capillary electrophoresis, Southern Blot, immunoassays, immunohistochemistry, ELISA, flow cytometry, Western blot, HPLC, and mass spectrometry.

The term “epitope” means a protein determinant capable of specific binding to an antibody, and/or directly involved in such a binding. An epitope usually consists of chemically active surface groupings of molecules such as amino acids or sugar side chains and usually has specific three dimensional structural characteristics, as well as specific charge characteristics. Conformational and non-conformational epitopes are distinguished in that the binding to the former but not the latter is lost in the presence of denaturing solvents. According to the invention, “epitopes” encompass conformational and non-conformational epitopes.

The term “eculizumab epitope” refers to the portions of the C5 protein, e.g. its amino acids, that are capable of being bound by eculizumab, and/or directly involved in such binding, wherein eculizumab binding induces a dysregulation of C5 activation. The eculizumab epitope contains the amino acid Arg at position 885 (Arg885), that is found within the MG7 domain of C5.

The term “antibody” as used herein includes whole antibodies and any antigen binding fragment (i.e. “antigen-binding portion”) or single chains thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen binding portion” of an antibody, as used herein, refers to one or more fragments of antibody that retain the ability to specifically bind to a given antigen (e.g., C5). Antigen binding functions of an antibody can be performed by fragments of an antibody. Examples of binding fragments encompassed within the term “antigen binding portion” of an antibody include a Fab fragment, a monovalent fragment consisting of the VL, VH, CL and CH1 domains; a F(ab)2 fragment, a bivalent fragment comprising two Fab fragments linked by a disulfide bridge at the hinge region; an Fd fragment consisting of the VH and CH1 domains; an Fv fragment consisting of the VL and VH domains of a single arm of an antibody; a single domain antibody (dAb) fragment (Ward et al., (1989) Nature 341: 544-546), which consists of a VH domain; and an isolated complementarity determining region (CDR).

Furthermore, although the two domains of the Fv fragment, VL and VH, are coded for by separate genes, they can be joined, using recombinant methods, by an artificial peptide linker that enables them to be made as a single protein chain in which the VL and VH regions pair to form monovalent molecules (known as single chain Fv (scFv); see, e.g., Bird et al., (1988) Science 242:423-426; and Huston et al., (1988) Proc. Natl. Acad. Sci. 85:5879-5883). Such single chain antibodies include one or more “antigen binding portions” of an antibody. These antibody fragments are obtained using conventional techniques known to those of skill in the art, and the fragments are screened for utility in the same manner as are antibodies.

Antigen binding portions can also be incorporated into single domain antibodies, maxibodies, minibodies, intrabodies, diabodies, triabodies, tetrabodies, v-NAR and bis-scFv (see, e.g., Hollinger and Hudson, (2005) Nature Biotechnology 23(9): 1126-1136). Antigen binding portions of antibodies can be grafted into scaffolds based on polypeptides such as Fibronectin type III (Fn3) (see U.S. Pat. No. 6,703,199, which describes fibronectin polypeptide monobodies).

The present invention provides antibodies that are capable of inhibiting the C5-component of complement activation through specific binding to a C5 protein (e.g., human and/or cynomologus C5). Such anti-C5 antibodies can be characterized by various functional assays. For example, they can be characterized by their ability to inhibit red blood cell lysis in hemolytic assays, their affinity to a C5 protein (e.g. human and/or cynomolgus C5), their epitope binning, their resistance to proteolysis, and their ability to block the activation of complement, for example, their ability to inhibit MAC formation.

In one embodiment, the anti-C5 antibody of the invention targets an epitope of the complement C5 protein that is not affected by a mutation or polymorphism within the MG7 domain of the complement C5 protein or within the eculizumab epitope thereof. For example the anti-C5 antibody of the invention targets an epitope of the complement C5 protein (e.g. binds thereto) that is not affected by a p.Arg885 polymorphism, e.g. p.Arg885His or p.Arg885Cys.

In another embodiment, the antibody of the invention is defined by its capability to effectively bind to the C5 protein, while such binding to the C5 protein is not affected by a mutation or polymorphism within the MG7 domain of the complement C5 protein or within the eculizumab epitope. For example the anti-C5 antibody of the invention is capable of effectively binding to a C5 protein that contains a p.Arg885 polymorphism, e.g. p.Arg885His or p.Arg885Cys.

The anti-C5 antibody according to the invention can target an epitope within the complement C5 protein that is located remotely from the MG7 domain of the C5 protein, the eculizumab epitope (including conformational epitope) or Arg885.

In another embodiment, the anti-C5 antibody of the invention targets an epitope within the C5 protein that does not include any known N-linked glycosylation site.

In one embodiment the anti-C5 antibody of the invention binds the C5 protein at, or close to, the CUB domain of the protein, e.g. at the interface of the CUB and TED/C5d domains of the C5 protein.

In one embodiment, the anti-C5 antibody to be administered is tesidolumab, which is described in Intl. Pat. Appl. No. WO 2010/015608, “Compositions and Methods for Antibodies Targeting Complement Protein C5” and U.S. Pat. No. 8,241,628, which are incorporated by reference. The CDR sequences of tesidolumab are included herein in Table 1: HCDR1 sequence (SEQ ID NO 5), HCDR2 sequence (SEQ ID NO. 6), HCDR3 sequence (SEQ ID NO. 7), LCDR1 sequence SEQ ID NO. 8), LCDR2 sequence (SEQ ID NO. 9), and LCDR3 sequence (SEQ ID NO. 10), as defined under the Kabat definition.

In another embodiment, the anti-C5 antibody to be administered is any antibody having the CDR sequences of tesidolumab, as described in SEQ ID NOs. 5-10. In another embodiment, the anti-C5 antibody to be administered specifically binds to the same epitope as tesidolumab.

Additional antibodies can therefore be identified based on their ability to cross-compete (e.g., to competitively inhibit the binding of, in a statistically significant manner) with the other antibodies disclosed herein in C5 binding assays e.g. a competition binding assay. The ability of a test antibody to inhibit the binding of antibodies of the present invention to a C5 protein (e.g., human and/or cynomolgus C5) demonstrates that the test antibody can compete with that antibody for binding to C5; such an antibody may, according to non-limiting theory, bind to the same or a related (e.g., a structurally similar or spatially proximal) epitope on the C5 protein as the antibody with which it competes. In a certain embodiment, the antibody that binds to the same epitope on C5 as the antibodies of the present invention is a human monoclonal antibody. Such human monoclonal antibodies can be prepared and isolated as described herein.

Known competition binding assays can be used to assess competition of a C5-binding antibody with the reference C5-binding antibody for binding to a C5 protein. These include, e.g., solid phase direct or indirect radioimmunoassay (RIA), solid phase direct or indirect enzyme immunoassay (EIA), sandwich competition assay (Stahli et al., (1983) Methods in Enzymology 9:242-253); solid phase direct biotin-avidin EIA (Kirkland et al., (1986) J. Immunol. 137:3614-3619); solid phase direct labeled assay, solid phase direct labeled sandwich assay; solid phase direct label RIA using 1-125 label (Morel et al., (1988) Molec. Immunol. 25:7-15); solid phase direct biotin-avidin EIA (Cheung et al., (1990) Virology 176:546-552); and direct labeled RIA (Moldenhauer et al., (1990) Scand. J. Immunol. 32:77-82). Typically, such an assay involves the use of purified antigen bound to a solid surface or cells bearing either of these, an unlabeled test C5-binding antibody and a labelled reference antibody. Competitive inhibition is measured by determining the amount of label bound to the solid surface or cells in the presence of the test antibody. Usually the test antibody is present in excess. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

To determine if the selected C5-binding monoclonal antibodies bind to unique epitopes, each antibody can be biotinylated using commercially available reagents (e.g., reagents from Pierce, Rockford, Ill. USA). Competition studies using unlabeled monoclonal antibodies and biotinylated monoclonal antibodies can be performed using a C5 polypeptide coated-ELISA plates. Biotinylated monoclonal antibody binding can be detected with a strep-avidin-alkaline phosphatase probe. To determine the isotype of a purified C5-binding antibody, isotype ELISAs can be performed. For example, wells of microtiter plates can be coated with 1 μg/ml of anti-human IgG overnight at 4° C. After blocking with 1% BSA, the plates are reacted with 1 μg/ml or less of the monoclonal C5-binding antibody or purified isotype controls, at ambient temperature for one to two hours. The wells can then be reacted with either human IgG- or human IgM-specific alkaline phosphatase-conjugated probes. Plates are then developed and analyzed so that the isotype of the purified antibody can be determined.

To demonstrate binding of monoclonal C5-binding antibodies to live cells expressing a C5 polypeptide, flow cytometry can be used. Briefly, cell lines expressing C5 (grown under standard growth conditions) can be mixed with various concentrations of a C5-binding antibody in PBS containing 0.1% BSA and 10% fetal calf serum, and incubated at 37° C. for 1 hour. After washing, the cells are reacted with fluorescein-labeled anti-human IgG antibody under the same conditions as the primary antibody staining. The samples can be analyzed by FACScan (BD Biosciences, San Jose, USA) using light and side scatter properties to gate on single cells. An alternative assay using fluorescence microscopy may be used (in addition to or instead of) the flow cytometry assay. Cells can be stained exactly as described above and examined by fluorescence microscopy. This method allows visualization of individual cells, but may have diminished sensitivity depending on the density of the antigen.

C5-binding antibodies of the invention can be further tested for reactivity with a C5 polypeptide or antigenic fragment by Western blotting. Briefly, purified C5 polypeptides or fusion proteins, or cell extracts from cells expressing C5 can be prepared and subjected to sodium dodecyl sulfate polyacrylamide gel electrophoresis. After electrophoresis, the separated antigens are transferred to nitrocellulose membranes, blocked with 10% fetal calf serum, and probed with the monoclonal antibodies to be tested. Human IgG binding can be detected using anti-human IgG alkaline phosphatase and developed with BCIP/NBT substrate tablets (Sigma Chem. Co., St. Louis, Mo. USA).

The present invention provides an anti-C5 antibody capable of inhibiting complement activation in a patient who has a mutation or polymorphism within the MG7 domain of the C5 protein, e.g. a mutation or polymorphism within the eculizumab epitope, e.g. a p.Arg885 polymorphism. In one embodiment, the present invention relates to an anti-C5 antibody or antigen binding fragment thereof for use in the treatment of a complement related disease or disorder, e.g. a C5 complement related disease or disorder, in a patient who has a p.Arg885 polymorphism in complement C5 protein, wherein said anti-C5 antibody is capable of inhibiting the complement pathway in said patient who has a p.Arg885 polymorphism.

The suitability of an anti-C5 antibody for use in the treatment of a complement related disease or disorder, e.g. a C5 complement related disease or disorder in a patient who has a mutation or polymorphism within the MG7 domain of the C5 protein, e.g. a mutation or polymorphism within the eculizumab epitope, e.g. a p.Arg885 polymorphism in complement C5 protein, can be tested with such assays as hemolysis assay or binding affinity assay. For example, to determine a pharmacodynamic response to an anti-C5 antibody, the capacity of the patients' serum to lyse antibody-sensitized chicken erythrocytes in a human serum-complement hemolytic assay can be measured (Hillmen et al., (2004) N Engl J Med. 350:552-9). According to Nishimura, less than 20% residual hemolysis is indicative of complete blockade of hemolysis in this assay system (Nishimura et al., (2014) New Engl J Med. 370:7). Binding of an anti-C5 antibody to C5 and different variants of C5 can be detected using binding affinity assay. Surface-plasmon-resonance analysis (Biacore 3000) can be used to assess the binding of an anti-C5 antibody to C5 with the use of an antihuman IgG (Fc) capture method described in Nishimura et al., supra, which is incorporated herein by reference.

In one embodiment, an anti-C5 antibody capable of inhibiting the complement pathway in a patient who has a mutation or polymorphism within the MG7 domain of the C5 protein or within the eculizumab epitope, e.g. a p.Arg885 polymorphism, is human anti-C5 antibody. The term “human antibody”, as used herein, is intended to include antibodies having variable regions in which both the framework and CDR regions are derived from sequences of human origin. Furthermore, if the antibody contains a constant region, the constant region also is derived from such human sequences, e.g. human germline sequences, or mutated versions of human germ line sequences. The human antibodies of the invention may include amino acid residues not encoded by human sequences (e.g., mutations introduced by random or site-specific mutagenesis in vitro or by somatic mutation in vivo). In certain embodiments, said antibody is a fully human Fc-silent IgG1/lambda monoclonal antibody that targets C5, such as tesidolumab. In a preferred embodiment, the present invention relates to the anti-C5 antibody tesidolumab for use in the prophylaxis or treatment of a C5 complement related disease or disorder, e.g. PNH or aHUS, in a patient who has a mutation or polymorphism within the MG7 domain of the C5 protein or within the eculizumab epitope, e.g. a p.Arg885 polymorphism in complement C5 protein.

In one embodiment, the present invention relates to an anti-C5 antibody having a binding epitope outside or remote from MG7 domain of the C5 protein. In another embodiment, the present invention relates to an anti-C5 antibody having a binding epitope remote from Arg885 or not overlapping with Arg885 position. C5 neutralization by said anti-C5 antibody is not affected by the Arg885 polymorphism observed in eculizumab non-responders, and thus said antibody is suitable for the present invention. Examples of an anti-C5 antibody having a binding epitope remote from Arg885 include tesidolumab or N19-8. In a preferred embodiment, the present invention relates to tesidolumab.

The invention is useful for treating human patients with a complement related disease or disorder, e.g. a C5 complement related disease or disorder. The terms “individual”, “host”, “subject”, and “patient” are used interchangeably to refer to an animal that is the object of treatment, observation and/or experiment. In general, such individual, host, subject or patient is a human, though other mammals are within the scope of the invention.

The term “treating” includes the administration of compositions or antibodies to prevent or delay the onset of the symptoms, complications, or biochemical indicia of a disease, alleviating the symptoms or arresting or inhibiting further development of the disease, condition, or disorder. Treatment may be prophylactic (to prevent or delay the onset of the disease, or to prevent the manifestation of clinical or subclinical symptoms thereof) or therapeutic suppression or alleviation of symptoms after the manifestation of the disease.

The term “a C5 complement related disease or disorder”, as used herein, refers to a disease or a disorder, wherein unregulated C5 function can result in disease phenotypes, for example due to dysregulated C5-activation, e.g. increased C5-activation.

Examples of known complement related diseases or disorders include: neurological disorders, multiple sclerosis, stroke, Guillain Barre Syndrome, traumatic brain injury, Parkinson's disease, Alzheimer's disease, disorders of inappropriate or undesirable complement activation, hemodialysis complications, interleukin-2 induced toxicity during IL-2 therapy, inflammatory disorders, inflammation of autoimmune diseases, Crohn's disease, adult respiratory distress syndrome, thermal injury including burns or frostbite, post-ischemic reperfusion conditions, Barraquer-Simons Syndrome, myocardial infarction, balloon angioplasty, post-pump syndrome in cardiopulmonary bypass or renal bypass, hemodialysis, renal ischemia, mesenteric artery reperfusion after acrotic reconstruction, infectious disease or sepsis, immune complex disorders and autoimmune diseases, rheumatoid arthritis, systemic lupus erythematosus (SLE), SLE nephritis, proliferative nephritis, hemolytic anemia, and myasthenia gravis. In addition, other known complement related disease are lung disease and disorders such as dyspnea, hemoptysis, ARDS, asthma, chronic obstructive pulmonary disease (COPD), emphysema, pulmonary embolisms and infarcts, pneumonia, fibrogenic dust diseases, inert dusts and minerals (e.g., silicon, coal dust, beryllium, and asbestos), pulmonary fibrosis, organic dust diseases, chemical injury (due to irritant gasses and chemicals, e.g., chlorine, phosgene, sulfur dioxide, hydrogen sulfide, nitrogen dioxide, ammonia, and hydrochloric acid), smoke injury, thermal injury (e.g., burn, freeze), allergy, bronchoconstriction, hypersensitivity pneumonitis, parasitic diseases, Goodpasture's Syndrome, pulmonary vasculitis, immune complex associated inflammation, aHUS, glomerulonephritis, bullous pemphigoid and membranoproliferative glomerulonephritis Type II (MPGN II), Geographic Atrophy (GA), neuromyelitis optica (NMO) and myasthenia gravis (MG).

In a specific embodiment, examples of known C5 complement related diseases or disorders include Geographic Atrophy (GA), Guillain Bane Syndrome, myasthenia gravis, SLE nephritis, proliferative nephritis, asthma, rheumatoid arthritis, sepsis: Paroxysmal Nocturnal Hemoglobinuria (PNH), atypical Hemolytic Uremic syndrome (aHUS) and Age-related Macular Degeneration (AMD).

PNH is a life-threatening disease of the blood and is characterized by, among other things, abnormal hematopoiesis, complement-mediated intravascular hemolysis, and a propensity for thrombosis. PNH arises as a consequence of clonal expansion of hematopoietic stem cells that have acquired a somatic mutation in the gene encoding phosphatidylinositol glycan anchor biosynthesis class A (PIGA), which encodes an enzyme that is necessary for the initial step of glycosylphosphatidylinositol (GPI) anchor biosynthesis. The resulting hematopoietic cells are deficient in glycosylphosphatidylinositol-anchored proteins, including the complement regulatory proteins CD55 and CD59; this accounts for the intravascular hemolysis that is the primary clinical manifestation of PNH. PNH frequently develops in association with disorders involving bone marrow failure, particularly aplastic anemia. Thrombosis is a major cause of PNH-associated morbidity and mortality.

Examples of disorders associated with PNH include anemia, thromboembolic events, smooth muscle dystonia, chronic kidney disease, erectile dysfunction, pulmonary hypertension and fatigue.

aHUS is an extremely rare, life-threatening, progressive disease that frequently has a genetic component. It is a disease associated with chronic risk of complement-mediated thrombotic microangiopathy (TMA) and life-threatening consequences. aHUS is defined as a disease that manifests with the clinical characteristics of TMA (thrombocytopenia, microangiopathic hemolysis and symptoms of organ dysfunction) and it affects adults as well as children.

Age-related Macular Degeneration (AMD) is a medical disorder predominantly found in the elderly in which the center of the inner lining of the eye, known as the macula area of the retina, suffers thinning, atrophy, and in some cases, bleeding. This can result in loss of central vision, which entails inability to see fine details, to read, or to recognize faces. Pathogenesis of new choroidal vessel formation is poorly understood, but factors such as inflammation, ischemia, and local production of angiogenic factors are thought to be important. The advanced form of the disease is divided between a “wet” (neovascular) form and a “dry” (geographic atrophy) form.

Geographic atrophy (GA) is an advanced atrophic form of dry AMD. GA is characterized by loss of photoreceptors, retinal pigment epithelium (RPE), and choriocapillaris within the macula.

In a preferred embodiment, a C5 complement related disease or disorder is PNH.

In one aspect, the present invention relates to an anti-C5 antibody or antigen binding fragment for use as a medicament in a method comprising administering an effective amount of an anti-C5 antibody capable of inhibiting the complement pathway in a patient who has a mutation or polymorphism within the MG7 domain of the C5 protein or within the eculizumab epitope, e.g. a p.Arg885 polymorphism, to said patient.

In a further aspect, the present invention relates to an anti-C5 antibody or antigen binding fragment for use in a method of preventing or treating a complement related disease or disorder, e.g. a C5 complement related disease or disorder, in a patient who has a mutation or polymorphism within the MG7 domain of the C5 protein or within the eculizumab epitope, e.g. a p.Arg885 polymorphism in complement C5 protein, wherein the method comprises administering an effective amount of an anti-C5 antibody capable of inhibiting the complement activation in said patient. In particular it relates to an anti-C5 antibody or antigen binding fragment for use in a method of preventing or treating a C5 complement related disease or disorder in a patient who has a p.Arg885 polymorphism in complement C5 protein, wherein the method comprises administering an effective amount of said anti-C5 antibody capable of inhibiting the complement activation in said patient.

In yet a further aspect, the present invention relates to a method of preventing or treating a complement related disease or disorder, e.g. a C5 complement related disease or disorder, in a patient in need thereof, wherein such patient has a mutation or polymorphism within the MG7 domain of the C5 protein or within the eculizumab epitope, e.g. a p.Arg885 polymorphism, comprising administering an effective amount of an anti-C5 antibody or antigen binding fragment capable of inhibiting the complement activation to said patient.

The term “administering” encompasses administration of an anti-C5 antibody or antigen binding fragment of the present invention, preferably tesidolumab, e.g. as multiple intravitreal doses in ophthalmic diseases. The term “administering” also encompasses administration of an anti-C5 antibody or antigen binding fragment of the present invention, preferably tesidolumab, in single and multiple intravenous (IV) doses in C5 related diseases such as PNH or aHUS. The term “an effective amount” or “therapeutically effective amount” of an anti-C5 antibody or antigen binding fragment thereof refers to an amount of the anti-C5 antibody or antigen binding fragment of the present disclosure that will elicit the biological or medical response of a subject, for example, reduction or inhibition of a protein activity, or ameliorate symptoms, alleviate conditions, slow or delay disease progression, or prevent a disease, etc. The term “effective amount” or “therapeutically effective amount” is defined herein to refer to an amount sufficient to provide an observable improvement over the baseline clinically observable signs and symptoms of the condition treated.

In one embodiment, the present invention relates to an anti-C5 antibody or antigen binding fragment thereof, preferably tesidolumab, for use in a method of prevention or treatment of PNH or aHUS.

According to the invention, the dose of the anti-C5 antibody or antigen binding fragment to be administered, e.g. tesidolumab, is between 10 mg/kg and 30 mg/kg, e.g. 15 mg/kg, 20 mg/kg, 25 mg/kg.

In certain embodiments, the anti-C5 antibody of the invention, e.g. tesidolumab, is administered 1, 2, 3, 4, 5, 6 or more times, during the treatment duration. For example, it is administered from 1 to 3, 1 to 4, 2 to 4, 2 to 5, 2 to 6, 3 to 6, 4 to 6, 6 to 8, or more times.

In some embodiments, the anti-C5 antibody of the invention, e.g. tesidolumab, is administered at least weekly, at least every two weeks, at least monthly.

The anti-C5 antibody of the invention, e.g. tesidolumab, can be administered over the period of at least 6 weeks, at least 9 weeks, at least 3 months, at least 6 months, at least 9 months, at least one year, lifelong.

In one embodiment, there is provided an anti-C5 antibody or antigen binding fragment thereof, e.g. tesidolumab, for use in prevention or treatment of PNH or aHUS, wherein said anti-C5 antibody is administered at a dose of at least 20 mg/kg weekly or every two weeks, for a period of at least one week, e.g. at least one month, e.g. at least 6 weeks, e.g. 3 months, e.g. 6 months, e.g. 9 months, e.g. one year, e.g. lifelong. Said antibody can be administered repeatedly at a dose of at least 20 mg/kg and at the interval between two administrations of not more than one month, e.g. is 2 weeks. Said antibody can be administered during at least 3 months, e.g. 6 months, e.g. 9 months, e.g. one year, e.g. lifelong.

In a further embodiment, an anti-C5 antibody or antigen binding fragment of the present invention, e.g. tesidolumab, for use in treatment of PNH, wherein said anti-C5 antibody is administered at a dose of at least 20 mg/kg weekly for a period of at least 6 weeks to 6 months, and then is administered at a dose of at least 20 mg/kg every two weeks for at least 3 months, 6 months, 9 months, 1 year, lifelong.

In another embodiment, there is provided an anti-C5 antibody or antigen binding fragment thereof, e.g. tesidolumab, for use in prevention or treatment of aHUS, wherein said anti-C5 antibody is administered at a dose of at least 20 mg/kg weekly or every two weeks, e.g. at least 30 mg/kg weekly or every two weeks. The administration can be for a period of at least one month, e.g. at least 6 weeks, e.g. 3 months, e.g. 6 months, e.g. 9 months, e.g. one year, e.g. lifelong.

The anti-C5 antibody of the invention, e.g. tesidolumab, can be administered repeatedly at a dose of at least 20 mg/kg, e.g. 30 mg/kg, at an interval between two administrations of not more than one month, e.g. 2 weeks. The anti-C5 antibody, e.g. tesidolumab, can be administered for at least 3 months, e.g. 6 months, e.g. 9 months, e.g. one year, e.g. lifelong.

In one embodiment, a patient is administered an anti-C5 antibody or antigen fragment thereof of the present invention, e.g. tesidolumab, wherein the patient is a naïve patient, e.g. said patient was not previously subjected to any an anti-C5 antibody or antigen fragment thereof treatment, in particular to eculizumab treatment (eculizumab-naïve patients). The population of eculizumab-naïve patients encompasses three different groups: (a) newly diagnosed cases; (b) diagnosed patients who do not have access to eculizumab and (c) early disease in which disease severity does not warrant treatment initiation, e.g. patients who did not have a thrombotic event.

In a yet further embodiment, a patient is administered an anti-C5 antibody or antigen fragment thereof of the present invention, e.g. tesidolumab, wherein the patient was previously administered an anti-C5 antibody or antigen fragment thereof, in particular eculizumab. In another embodiment, a patient is administered an anti-C5 antibody or antigen fragment thereof of the present invention, e.g. tesidolumab, wherein the patient was previously administered an anti-C5 antibody or antigen fragment thereof, in particular eculizumab, and wherein the patient is not responsive to said previous treatment, e.g. eculizumab treatment, in particular wherein the patient has an p.Arg885 polymorphism in complement C5 protein.

In one aspect, the present invention relates to use of an anti-C5 antibody or antigen binding fragment thereof, e.g. tesidolumab, for the manufacture of a medicament for the prophylaxis or treatment of a complement related disease or disorder, e.g. a C5 complement related disease or disorder, e.g. PNH or aHUS, in a patient who has a p.Arg885 polymorphism in complement C5 protein. In one embodiment, the present invention relates to use of an anti-C5 antibody or antigen binding fragment thereof for the manufacture of a medicament for the treatment of a C5 complement related disease or disorder in a patient who has a mutation or polymorphism within the MG7 domain of the C5 protein or within the eculizumab epitope, a p.Arg885 polymorphism in complement C5 protein, wherein said anti-C5 antibody is capable of inhibiting the complement activation in said patient, e.g. tesidolumab. For example, there is provided the use of tesidolumab or an antigen binding fragment thereof for the manufacture of a medicament for the prophylaxis or treatment of a C5 complement related disease or disorder, e.g. PNH or aHUS, in a patient who has a p.Arg885 polymorphism in complement C5 protein.

In one embodiment, the method of preventing or treating a complement related disease or disorder, e.g. a C5 complement related disease or disorder, further comprises the step of determining from a biological sample obtained from a patient whether the C5 complement protein of the patient comprises a mutation or polymorphism within the MG7 domain of the C5 protein or within the eculizumab epitope, e.g. a p.Arg885 polymorphism, wherein the biological sample is of tissue or fluid isolated from the patient.

The term “biological sample” as used herein, refers to a biological specimen taken by sampling so as to be representative of any other specimen taken from the source of the specimen. In one embodiment, a biological sample is tissue or fluid isolated from a patient.

In one aspect, the present invention relates to an anti-C5 antibody or antigen binding fragment for use in a method of treating a complement related disease or disorder, e.g. a C5 complement related disease or disorder, in a patient in need thereof, the method comprising: (a) taking a biological sample from the patient; (b) screening for a mutation or polymorphism in the gene encoding C5 of said patient; (c) determining whether the patient has either a mutation or polymorphism within the MG7 domain of the C5 protein, within the eculizumab epitope or has the p.Arg885 polymorphism in the C5 complement protein; (d) administering an effective amount of an anti-C5 antibody capable of inhibiting the complement activation in a patient who has at least a mutation or polymorphism detected under step (c), wherein the biological sample is of tissue or fluid isolated from the patient. In a preferred embodiment, said anti-C5 antibody is tesidolumab. In another embodiment, the mutation or polymorphism in the C5 protein is p.Arg885 polymorphism, e.g. p.Arg885His or p.Arg885Cys.

In another aspect, the present invention relates to an anti-C5 antibody or antigen binding fragment thereof for use in a method of treating PNH or aHUS, the method comprising: (a) determining from a biological sample obtained from a patient whether the patient has either a mutation or polymorphism within the MG7 domain of the C5 protein, within the eculizumab epitope or the p.Arg885 polymorphism in the C5 complement protein, wherein the biological sample is of tissue or fluid isolated from the patient; and (b) administering an effective amount of the anti-C5 antibody or antigen binding fragment thereof, e.g. tesidolumab or an antigen binding fragment thereof, to said patient.

TABLE 1
SEQUENCES

| SEQ ID NO. | Information | Sequence |
|---|---|---|
| 1 | Human C5 | MGLLGILCFLIFLGKTWGQEQTYVISAPKIFRVGAS |
| | protein | ENIVIQVYGYTEAFDATISIKSYPDKKFSYSSGHVH |
| | | LSSENKFQNSAILTIQPKQLPGGQNPVSYVYLEVVS |
| | | KHFSKSKRMPITYDNGFLFIHTDKPVYTPDQSVKVR |
| | | VYSLNDDLKPAKRETVLTFIDPEGSEVDMVEEIDHI |
| | | GIISFPDFKIPSNPRYGMWTIKAKYKEDFSTTGTAY |
| | | FEVKEYVLPHFSVSIEPEYNFIGYKNFKNFEITIKA |
| | | RYFYNKVVTEADVYITFGIREDLKDDQKEMMQTAMQ |
| | | NTMLINGIAQVTFDSETAVKELSYYSLEDLNNKYLY |
| | | IAVTVIESTGGFSEEAEIPGIKYVLSPYKLNLVATP |
| | | LFLKPGIPYPIKVQVKDSLDQLVGGVPVTLNAQTID |
| | | VNQETSDLDPSKSVTRVDDGVASFVLNLPSGVTVLE |
| | | FNVKTDAPDLPEENQAREGYRAIAYSSLSQSYLYID |
| | | WTDNHKALLVGEHLNIIVTPKSPYIDKITHYNYLIL |
| | | SKGKIIHFGTREKFSDASYQSINIPVTQNMVPSSRL |
| | | LVYYIVTGEQTAELVSDSVWLNIEEKCGNQLQVHLS |
| | | PDADAYSPGQTVSLNMATGMDSWVALAAVDSAVYGV |
| | | QRGAKKPLERVFQFLEKSDLGCGAGGGLNNANVFHL |
| | | AGLTFLTNANADDSQENDEPCKEILRPRRTLQKKIE |
| | | EIAAKYKHSVVKKCCYDGACVNNDETCEQRAARISL |
| | | GPRCIKAFTECCVVASQLRANISHKDMQLGRLHMKT |
| | | LLPVSKPEIRSYFPESWLWEVHLVPRRKQLQFALPD |
| | | SLTTWEIQGVGISNTGICVADTVKAKVFKDVFLEMN |
| | | IPYSVVRGEQIQLKGTVYNYRTSGMQFCVKMSAVEG |
| | | ICTSESPVIDHQGTKSSKCVRQKVEGSSSHLVTFTV |
| | | LPLEIGLHNINFSLETWFGKEILVKTLRVVPEGVKR |
| | | ESYSGVTLDPRGIYGTISRRKEFPYRIPLDLVPKTE |
| | | IKRILSVKGLLVGEILSAVLSQEGINILTHLPKGSA |
| | | EAELMSVVPVFYVFHYLETGNHWNIFHSDPLIEKQK |
| | | LKKKLKEGMLSIMSYRNADYSYSVWKGGSASTWLTA |
| | | FALRVLGQVNKYVEQNQNSICNSLLWLVENYQLDNG |
| | | SFKENSQYQPIKLQGTLPVEARENSLYLTAFTVIGI |
| | | RKAFDICPLVKIDTALIKADNFLLENTLPAQSTFTL |
| | | AISAYALSLGDKTHPQFRSIVSALKREALVKGNPPI |
| | | YRFWKDNLQHKDSSVPNTGTARMVETTAYALLTSLN |
| | | LKDINYVNPVIKWLSEEQRYGGGFYSTQDTINAIEG |
| | | LTEYSLLVKQLRLSMDIDVSYKHKGALHNYKMTDKN |
| | | FLGRPVEVLLNDDLIVSTGFGSGLATVHVTTVVHKT |
| | | STSEEVCSFYLKIDTQDIEASHYRGYGNSDYKRIVA |
| | | CASYKPSREESSSGSSHAVMDISLPTGISANEEDLK |
| | | ALVEGVDQLFTDYQIKDGHVILQLNSIPSSDFLCVR |
| | | FRIFELFEVGFLSPATFTVYEYHRPDKQCTMFYSTS |
| | | NIKIQKVCEGAACKCVEADCGQMQEELDLTISAETR |
| | | KQTACKPEIAYAYKVSITSITVENVFVKYKATLLDI |
| | | YKTGEAVAEKDSEITFIKKVTCTNAELVKGRQYLIM |
| | | GKEALQIKYNFSFRYIYPLDSLTWIEYWPRDTTCSS |
| | | CQAFLANLDEFAEDIFLNGC |
| 2 | Cynomolgus C5 | MGLLGILCFLIFLGKTWGQEQTYVISAPKIFRVGAS |
| | protein | ENIVIQVYGYTEAFDATISIKSYPDKKFSYSSGHVH |
| | (Macaca | LSSENKFQNSAVLTIQPKQLPGGQNQVSYVYLEVVS |
| | fascicularis) | KHFSKSKKIPITYDNGFLFIHTDKPVYTPDQSVKVR |
| | | VYSLNDDLKPAKRETVLTFIDPEGSEIDMVEEIDHI |
| | | GIISFPDFKIPSNPRYGMWTIQAKYKEDFSTTGTAF |
| | | FEVKEYVLPHFSVSVEPESNFIGYKNFKNFEITIKA |
| | | RYFYNKVVTEADVYITFGIREDLKDDQKEMMQTAMQ |
| | | NTMLINGIAQVTFDSETAVKELSYYSLEDLNNKYLY |
| | | IAVTVIESTGGFSEEAEIPGIKYVLSPYKLNLVATP |
| | | LFLKPGIPYSIKVQVKDALDQLVGGVPVTLNAQTID |
| | | VNQETSDLEPRKSVTRVDDGVASFVVNLPSGVTVLE |
| | | FNVKTDAPDLPDENQAREGYRAIAYSSLSQSYLYID |
| | | WTDNHKALLVGEYLNIIVTPKSPYIDKITHYNYLIL |
| | | SKGKIIHFGTREKLSDASYQSINIPVTQNMVPSSRL |
| | | LVYYIVTGEQTAELVSDSVWLNIEEKCGNQLQVHLS |
| | | PDADTYSPGQTVSLNMVTGMDSWVALTAVDSAVYGV |
| | | QRRAKKPLERVFQFLEKSDLGCGAGGGLNNANVFHL |
| | | AGLTFLTNANADDSQENDEPCKEIIRPRRMLQEKIE |
| | | EIAAKYKHLVVKKCCYDGVRINHDETCEQRAARISV |
| | | GPRCVKAFTECCVVASQLRANNSHKDLQLGRLHMKT |
| | | LLPVSKPEIRSYFPESWLWEVHLVPRRKQLQFALPD |
| | | SVTTWEIQGVGISNSGICVADTIKAKVFKDVFLEMN |
| | | IPYSVVRGEQVQLKGTVYNYRTSGMQFCVKMSAVEG |
| | | ICTSESPVIDHQGTKSSKCVRQKVEGSSNHLVTFTV |
| | | LPLEIGLQNINFSLETSFGKEILVKSLRVVPEGVKR |
| | | ESYSGITLDPRGIYGTISRRKEFPYRIPLDLVPKTE |
| | | IKRILSVKGLLVGEILSAVLSREGINILTHLPKGSA |
| | | EAELMSVVPVFYVFHYLETGNHWNIFHSDPLIEKRN |
| | | LEKKLKEGMVSIMSYRNADYSYSVWKGGSASTWLTA |
| | | FALRVLGQVHKYVEQNQNSICNSLLWLVENYQLDNG |
| | | SFKENSQYQPIKLQGTLPVEARENSLYLTAFTVIGI |
| | | RKAFDICPLVKINTALIKADTFLLENTLPAQSTFTL |
| | | AISAYALSLGDKTHPQFRSIVSALKREALVKGNPPI |
| | | YRFWKDSLQHKDSSVPNTGTARMVETTAYALLTSLN |
| | | LKDINYVNPIIKWLSEEQRYGGGFYSTQDTINAIEG |
| | | LTEYSLLVKQLRLNMDIDVAYKHKGPLHNYKMTDKN |
| | | FLGRPVEVLLNDDLVVSTGFGSGLATVHVTTVVHKT |
| | | STSEEVCSFYLKIDTQDIEASHYRGYGNSDYKRIVA |
| | | CASYKPSKEESSSGSSHAVMDISLPTGINANEEDLK |
| | | ALVEGVDQLFTDYQIKDGHVILQLNSIPSSDFLCVR |
| | | FRIFELFEVGFLSPATFTVYEYHRPDKQCTMFYSTS |
| | | NIKIQKVCEGATCKCIEADCGQMQKELDLTISAETR |
| | | KQTACNPEIAYAYKVIITSITTENVFVKYKATLLDI |
| | | YKTGEAVAEKDSEITFIKKVTCTNAELVKGRQYLIM |
| | | GKEALQIKYNFTFRYIYPLDSLTWIEYWPRDTTCS |
| | | SCQAFLANLDEFAEDIFLNGC |
| 3 | Arg885His | MGLLGILCFLIFLGKTWGQEQTYVISAPKIFRVGAS |
| | variant human C5 | ENIVIQVYGYTEAFDATISIKSYPDKKFSYSSGHVH |
| | protein | LSSENKFQNSAILTIQPKQLPGGQNPVSYVYLEVVS |
| | | KHFSKSKRMPITYDNGFLFIHTDKPVYTPDQSVKVR |
| | | VYSLNDDLKPAKRETVLTFIDPEGSEVDMVEEIDHI |
| | | GHSFPDFKIPSNPRYGMWTIKAKYKEDFSTTGTAYF |
| | | EVKEYVLPHFSVSIEPEYNFIGYKNFKNFEITIKAR |
| | | YFYNKVVTEADVYITFGIREDLKDDQKEMMQTAMQN |
| | | TMLINGIAQVTFDSETAVKELSYYSLEDLNNKYLYI |
| | | AVTVIESTGGFSEEAEIPGIKYVLSPYKLNLVATPL |
| | | FLKPGIPYPIKVQVKDSLDQLVGGVPVTLNAQTIDV |
| | | NQETSDLDPSKSVTRVDDGVASFVLNLPSGVTVLEF |
| | | NVKTDAPDLPEENQAREGYRAIAYSSLSQSYLYIDW |
| | | TDNHKALLVGEHLNIIVTPKSPYIDKITHYNYLILS |
| | | KGKIIHFGTREKFSDASYQSINIPVTQNMVPSSRLL |
| | | VYYIVTGEQTAELVSDSVWLNIEEKCGNQLQVHLSP |
| | | DADAYSPGQTVSLNMATGMDSWVALAAVDSAVYGVQ |
| | | RGAKKPLERVFQFLEKSDLGCGAGGGLNNANVFHLA |
| | | GLTFLTNANADDSQENDEPCKEILRPRRTLQKKIEE |
| | | IAAKYKHSVVKKCCYDGACVNNDETCEQRAARISLG |
| | | PRCIKAFTECCVVASQLRANISHKDMQLGRLHMKTL |
| | | LPVSKPEIRSYFPESWLWEVHLVPRRKQLQFALPDS |
| | | LTTWEIQGVGISNTGICVADTVKAKVFKDVFLEMNI |
| | | PYSVVRGEQIQLKGTVYNYRTSGMQFCVKMSAVEGI |
| | | CTSESPVIDHQGTKSSKCVHQKVEGSSSHLVTFTVL |
| | | PLEIGLHNINFSLETWFGKEILVKTLRVVPEGVKRE |
| | | SYSGVTLDPRGIYGTISRRKEFPYRIPLDLVPKTEI |
| | | KRILSVKGLLVGEILSAVLSQEGINILTHLPKGSAE |
| | | AELMSVVPVFYVFHYLETGNHWNIFHSDPLIEKQKL |
| | | KKKLKEGMLSIMSYRNADYSYSVWKGGSASTWLTAF |
| | | ALRVLGQVNKYVEQNQNSICNSLLWLVENYQLDNGS |
| | | FKENSQYQPIKLQGTLPVEARENSLYLTAFTVIGIR |
| | | KAFDICPLVKIDTALIKADNFLLENTLPAQSTFTLA |
| | | ISAYALSLGDKTHPQFRSIVSALKREALVKGNPPIY |
| | | RFWKDNLQHKDSSVPNTGTARMVETTAYALLTSLNL |
| | | KDINYVNPVIKWLSEEQRYGGGFYSTQDTINAIEGL |
| | | TEYSLLVKQLRLSMDIDVSYKHKGALHNYKMTDKNF |
| | | LGRPVEVLLNDDLIVSTGFGSGLATVHVTTVVHKTS |
| | | TSEEVCSFYLKIDTQDIEASHYRGYGNSDYKRIVAC |
| | | ASYKPSREESSSGSSHAVMDISLPTGISANEEDLKA |
| | | LVEGVDQLFTDYQIKDGHVILQLNSIPSSDFLCVRF |
| | | RIFELFEVGFLSPATFTVYEYHRPDKQCTMFYSTSN |
| | | IKIQKVCEGAACKCVEADCGQMQEELDLTISAETRK |
| | | QTACKPEIAYAYKVSITSITVENVFVKYKATLLDIY |
| | | KTGEAVAEKDSEITFIKKVTCTNAELVKGRQYLIMG |
| | | KEALQIKYNFSFRYIYPLDSLTWIEYWPRDTTCSSC |
| | | QAFLANLDEFAEDIFLNGC |
| 4 | Arg885Cys | MGLLGILCFLIFLGKTWGQEQTYVISAPKIFRVGAS |
| | variant human C5 | ENIVIQVYGYTEAFDATISIKSYPDKKFSYSSGHVH |
| | protein | LSSENKFQNSAILTIQPKQLPGGQNPVSYVYLEVVS |
| | | KHFSKSKRMPITYDNGFLFIHTDKPVYTPDQSVKVR |
| | | VYSLNDDLKPAKRETVLTFIDPEGSEVDMVEEIDHI |
| | | GHSFPDFKIPSNPRYGMWTIKAKYKEDFSTTGTAYF |
| | | EVKEYVLPHFSVSIEPEYNFIGYKNFKNFEITIKAR |
| | | YFYNKVVTEADVYITFGIREDLKDDQKEMMQTAMQN |
| | | TMLINGIAQVTFDSETAVKELSYYSLEDLNNKYLYI |
| | | AVTVIESTGGFSEEAEIPGIKYVLSPYKLNLVATPL |
| | | FLKPGIPYPIKVQVKDSLDQLVGGVPVTLNAQTIDV |
| | | NQETSDLDPSKSVTRVDDGVASFVLNLPSGVTVLEF |
| | | NVKTDAPDLPEENQAREGYRAIAYSSLSQSYLYIDW |
| | | TDNHKALLVGEHLNIIVTPKSPYIDKITHYNYLILS |
| | | KGKIIHFGTREKFSDASYQSINIPVTQNMVPSSRLL |
| | | VYYIVTGEQTAELVSDSVWLNIEEKCGNQLQVHLSP |
| | | DADAYSPGQTVSLNMATGMDSWVALAAVDSAVYGVQ |
| | | RGAKKPLERVFQFLEKSDLGCGAGGGLNNANVFHLA |
| | | GLTFLTNANADDSQENDEPCKEILRPRRTLQKKIEE |
| | | IAAKYKHSVVKKCCYDGACVNNDETCEQRAARISLG |
| | | PRCIKAFTECCVVASQLRANISHKDMQLGRLHMKTL |
| | | LPVSKPEIRSYFPESWLWEVHLVPRRKQLQFALPDS |
| | | LTTWEIQGVGISNTGICVADTVKAKVFKDVFLEMNI |
| | | PYSVVRGEQIQLKGTVYNYRTSGMQFCVKMSAVEGI |
| | | CTSESPVIDHQGTKSSKCVCQKVEGSSSHLVTFTVL |
| | | PLEIGLHNINFSLETWFGKEILVKTLRVVPEGVKRE |
| | | SYSGVTLDPRGIYGTISRRKEFPYRIPLDLVPKTEI |
| | | KRILSVKGLLVGEILSAVLSQEGINILTHLPKGSAE |
| | | AELMSVVPVFYVFHYLETGNHWNIFHSDPLIEKQKL |
| | | KKKLKEGMLSIMSYRNADYSYSVWKGGSASTWLTAF |
| | | ALRVLGQVNKYVEQNQNSICNSLLWLVENYQLDNGS |
| | | FKENSQYQPIKLQGTLPVEARENSLYLTAFTVIGIR |
| | | KAFDICPLVKIDTALIKADNFLLENTLPAQSTFTLA |
| | | ISAYALSLGDKTHPQFRSIVSALKREALVKGNPPIY |
| | | RFWKDNLQHKDSSVPNTGTARMVETTAYALLTSLNL |
| | | KDINYVNPVIKWLSEEQRYGGGFYSTQDTINAIEGL |
| | | TEYSLLVKQLRLSMDIDVSYKHKGALHNYKMTDKNF |
| | | LGRPVEVLLNDDLIVSTGFGSGLATVHVTTVVHKTS |
| | | TSEEVCSFYLKIDTQDIEASHYRGYGNSDYKRIVAC |
| | | ASYKPSREESSSGSSHAVMDISLPTGISANEEDLKA |
| | | LVEGVDQLFTDYQIKDGHVILQLNSIPSSDFLCVRF |
| | | RIFELFEVGFLSPATFTVYEYHRPDKQCTMFYSTSN |
| | | IKIQKVCEGAACKCVEADCGQMQEELDLTISAETRK |
| | | QTACKPEIAYAYKVSITSITVENVFVKYKATLLDIY |
| | | KTGEAVAEKDSEITFIKKVTCTNAELVKGRQYLIMG |
| | | KEALQIKYNFSFRYIYPLDSLTWIEYWPRDTTCSSC |
| | | QAFLANLDEFAEDIFLNGC |
| 5 | tesidolumab | SYAIS |
| | HCDR1 | |
| 6 | tesidolumab | GIGPFFGTANYAQKFQG |
| | HCDR2 | |
| 7 | tesidolumab | DTPYFDY |
| | HCDR3 | |
| 8 | tesidolumab | SGDSIPNYYVY |
| | LCDR1 | |
| 9 | tesidolumab | DDSNRPS |
| | LCDR2 | |
| 10 | tesidolumab | QSFDSSLNAEV |
| | LCDR3 | |
| 11 | tesidolumab VH | EVQLVQSGAEVKKPGSSVKVSCKASGGTFSSYAIS |
| | | WVRQAPGQGLEWMGGIGPFFGTANYAQKFQGRVTI |
| | | TADESTSTAYMELSSLRSEDTAVYYCARDTPYFDY |
| | | WGQGTLVTVSS |
| 12 | tesidolumab VL | SYELTQPLSVSVALGQTARITCSGDSIPNYYVYWYQ |
| | | QKPGQAPVLVIYDDSNRPSGIPERFSGSNSGNTATL |
| | | TISRAQAGDEADYYCQSFDSSLNAEVFGGGTKLTVL |
| 13 | tesidolumab HC | EVQLVQSGAEVKKPGSSVKVSCKASGGTFSSYAISW |
| | | VRQAPGQGLEWMGGIGPFFGTANYAQKFQGRVTITA |
| | | DESTSTAYMELSSLRSEDTAVYYCARDTPYFDYWGQ |
| | | GTLVTVSSASTKGPSVFPLAPSSKSTSGGTAALGCL |
| | | VKDYFPEPVTVSWNSGALTSGVHTFPAVLQSSGLYS |
| | | LSSVVTVPSSSLGTQTYICNVNHKPSNTKVDKRVEP |
| | | KSCDKTHTCPPCPAPEAAGGPSVFLFPPKPKDTLMI |
| | | SRTPEVTCVVVDVSHEDPEVKFNWYVDGVEVHNAKT |
| | | KPREEQYNSTYRVVSVLTVLHQDWLNGKEYKCKVSN |
| | | KALPAPIEKTISKAKGQPREPQVYTLPPSREEMTKN |
| | | QVSLTCLVKGFYPSDIAVEWESNGQPENNYKTTPPV |
| | | LDSDGSFFLYSKLTVDKSRWQQGNVFSCSVMHEALH |
| | | NHYTQKSLSLSPGK |
| 14 | tesidolumab LC | SYELTQPLSVSVALGQTARITCSGDSIPNYYVYWYQ |
| | | QKPGQAPVLVIYDDSNRPSGIPERFSGSNSGNTATL |
| | | TISRAQAGDEADYYCQSFDSSLNAEVFGGGTKLTVL |
| | | GQPKAAPSVTLFPPSSEELQANKATLVCLISDFYPG |
| | | AVTVAWKADSSPVKAGVETTTPSKQSNNKYAASSYL |
| | | SLTPEQWKSHRSYSCQVTHEGSTVEKTVAPTECS |
| 15 | eculizumab | NYWIQ |
| | HCDR1 | |
| 16 | eculizumab | EILPGSGSTEYTENFKD |
| | HCDR2 | |
| 17 | eculizumab | YFFGSSPNWYFDV |
| | HCDR3 | |
| 18 | eculizumab | GASENIYGALN |
| | LCDR1 | |
| 19 | eculizumab | GATNLAD |
| | LCDR2 | |
| 20 | eculizumab | QNVLNTPLT |
| | LCDR3 | |
| 21 | eculizumab VH | QVQLVQSGAEVKKPGASVKVSCKASGYIFSNYWIQW |
| | | VRQAPGQGLEWMGEILPGSGSTEYTENFKDRVTMTR |
| | | DTSTSTVYMELSSLRSEDTAVYYCARYFFGSSPNWY |
| | | FDVWGQGTLVTVSSA |
| 22 | eculizumab VL | MDMRVPAQLLGLLLLWLRGARCDIQMTQSPSSLSAS |
| | | VGDRVTITCGASENIYGALNWYQQKPGKAPKLLIYG |
| | | ATNLADGVPSRFSGSGSGTDFTLTISSLQPEDFATY |
| | | YCQNVLNTPLTFGQGTKVEIKRT |
| 23 | eculizumab HC | QVQLVQSGAEVKKPGASVKVSCKASGYIFSNYWIQW |
| | | VRQAPGQGLEWMGEILPGSGSTEYTENFKDRVTMTR |
| | | DTSTSTVYMELSSLRSEDTAVYYCARYFFGSSPNWY |
| | | FDVWGQGTLVTVSSASTKGPSVFPLAPCSRSTSEST |
| | | AALGCLVKDYFPEPVTVSWNSGALTSGVHTFPAVLQ |
| | | SSGLYSLSSVVTVPSSNFGTQTYTCNVDHKPSNTKV |
| | | DKTVERKCCVECPPCPAPPVAGPSVFLFPPKPKDTL |
| | | MISRTPEVTCVVVDVSQEDPEVQFNWYVDGVEVHNA |
| | | KTKPREEQFNSTYRVVSVLTVLHQDWLNGKEYKCKV |
| | | SNKGLPSSIEKTISKAKGQPREPQVYTLPPSQEEMT |
| | | KNQVSLTCLVKGFYPSDIAVEWESNGQPENNYKTTP |
| | | PVLDSDGSFFLYSRLTVDKSRWQEGNVFSCSVMHEA |
| | | LHNHYTQKSLSLSLGK |
| 24 | eculizumab LC | MDMRVPAQLLGLLLLWLRGARCDIQMTQSPSSLSAS |
| | | VGDRVTITCGASENIYGALNWYQQKPGKAPKLLIYG |
| | | ATNLADGVPSRFSGSGSGTDFTLTISSLQPEDFATY |
| | | YCQNVLNTPLTFGQGTKVEIKRTVAAPSVFIFPPSD |
| | | EQLKSGTASVVCLLNNFYPREAKVQWKVDNALQSGN |
| | | SQESVTEQDSKDSTYSLSSTLTLSKADYEKHKVYAC |
| | | EVTHQGLSSPVTKSFNRGEC |

The following Examples illustrate the invention described above, but are not, however, intended to limit the scope of the invention in any way. Other test models known as such to the person skilled in the pertinent art can also determine the beneficial effects of the claimed invention.

### EXAMPLES

### Example 1: Crystallization of the Tesidolumab Fab in Complex with Human C5

Tesidolumab is a human monoclonal antibody that binds to human and cynomolgus (Macaca fascicularis) complement C5 with picomolar affinity, thereby preventing C5 activation and the release of C5a and C5b. A detailed analysis of tesidolumab in complex with human C5 has been carried out.

### Methods:

### Expression and Purification of the Tesidolumab Fab

Tesidolumab Fab was cloned and expressed in TG1F⁻ E. coli cells (ACE25090). Frozen cell pellets were suspended in 150 ml lysis buffer and homogenized (Lysis buffer: 20 mM NaH₂PO₄, 10 mM imidazole, 500 mM NaCl pH 7.4, with 1 tablet of EDTA-free cOmplete™ protease inhibitor cocktail (Roche) per 50 ml buffer, 4500 of 1.0M MgCl₂ and 150 of benzonase (Novagen)). After centrifugation (30 min at 16,000 g, 4° C.), the supernatant was sterile filtered (0.2 μm Stericup filter) and loaded (2.5 ml/min) on a 5 ml HisTrap HP column (GE Healthcare, 17-5247-01) equilibrated with lysis buffer. After two washing steps at 20 mM and then 50 mM imidazole, the Fab was eluted by a 100 ml gradient from 50 mM to 500 mM imidazole. The eluate was collected in 5 ml fractions and analyzed by SDS-PAGE using 10% Bis-Tris gel (NuPage, Invitrogen). Selected fractions were pooled, concentrated to 5 ml at 4° C. by ultrafiltration (Amicon Ultra-15 3 k concentrator) and loaded on a Superdex75 column equilibrated with 10 mM Tris-HCl pH 7.5, 25 mM NaCl. Collected fractions were analyzed as before by SDS-PAGE, pooled and concentrated by ultrafiltration. The Fab was then further purified over a MonoQ HR 10/10 cation exchange column equilibrated with 50 mM Tris-HCl pH 8.0, using a 0.0-1.0M NaCl gradient for elution. Pooled fractions were concentrated and again loaded in several runs on a Superdex75 300 GL column with isocratic elution in 10 mM Tris-HCl pH 7.5, 25 mM NaCl.

Preparation and Purification of the Tesidolumab Fab Complex with Human C5

Human complement protein C5 was purchased from Complement Technology, Inc. (cat. no. A120 Lot 16a) and used without further purification. A 2.5-fold molar excess of the tesidolumab Fab was added to human C5 and the complex was purified by size-exclusion chromatography with a S300 Sephacryl 16/60 column equilibrated with 10 mM Tris pH 7.4, 25 mM NaCl.

Crystallization of the Tesidolumab Fab Complex with Human C5

The tesidolumab Fab complex with human C5 in 10 mM Tris pH 7.4, 25 mM NaCl was concentrated to 17.8 mg/ml by ultrafiltration and submitted to crystallization screening at 20° C. Crystallization conditions were initially identified by sitting drop vapor diffusion in 96-well Innovadyne SD2 plates (CHBS_19814_G12_1). Larger crystals (CHBS_20088_B3_1) were then grown in 20 drops by the technique of vapor diffusion in hanging drop using 24-well VDX plates (Hampton Research).

X-Ray Data Collection and Structure Determination of the Tesidolumab Fab Complex with Human C5

Two diffraction data sets were collected from crystals of the tesidolumab Fab complex. Both data sets were processed with XDS and XSCALE (Kabsch 1993) as before. The second data set was later reprocessed with the Jul. 4, 2012 version of XSCALE in order to include during refinement weak diffraction data beyond 4.1 Å resolution which still had a significant percentage of correlation statistic CC* (Karplus and Diederichs 2012).

Data set 1 was collected at beamline X06DA (PXIII) of the Swiss Light Source (Paul Scherrer Institute, Switzerland), equipped with a MAR CCD 225 mm detector, and using X-rays of 1.00000 Å wavelength. The crystal used in this experiment was directly flash cooled into liquid nitrogen. In total, 180 images of 1.0 deg oscillation each were recorded at a crystal to detector distance of 380 mm. This diffraction data set was to a resolution of 4.5 Å.

Data set 2 was collected at beamline X10SA (PXII) of the Swiss Light Source (Paul Scherrer Institute, Switzerland), equipped with a Pilatus pixel detector, and using X-rays of 1.00000 Å wavelength. Prior to flash cooling into liquid nitrogen, the crystal used in this experiment was briefly soaked in the mother liquor supplemented with 10 μM CdCl₂. In total, 720 images of 0.25 deg oscillation each were recorded at a crystal to detector distance of 600 mm. This diffraction data set was to a resolution of 4.1 Å.

The structure was solved by molecular replacement using multiple Phaser runs (McCoy et al 2007). When full-length human C5 (PDB entry 3CU7, chain A; Fredslund et al 2008) was used as a search model, no molecular replacement solution could be found. A second Phaser run using full-length C5 without the C345C domain was also unsuccessful. In sharp contrast, a clear molecular replacement solution in space group P4₃ was readily found when the C5 β-chain was used as a search model (TFZ-score=8.2). With the solution for the C5 β-chain fixed, a clear molecular replacement solution was then found for the α-chain without the C345C domain (TFZ-score=22.8). A clear solution for the C345C domain was obtained from a subsequent Phaser run (TFZ-score=13.5). Then, the variable and constant domains of the Tesidolumab Fab (structure refined at 2.1 Å resolution from crystal form 2) were used as search models. The V_{L}/V_{H} fragment gave a very clear molecular replacement solution (TFZ-score=23.5). Although the C_{L}/C_{H1} domain gave a weaker signal (TFZ-score=6.6), a meaningful solution (as judged from the connectivity to the previously positioned V_{L}/V_{H} domain) was readily found. The molecular replacement calculations were first performed with the 4.5 Å diffraction data set and were then repeated when 4.1 Å data became available, leading to the same overall solution.

The complete molecular replacement model was inspected in COOT (Emsley et al 2010) and was refined with Buster 2.11.2 (Bricogne et al 2011) against all diffraction data to 3.3 Å resolution. Because of the limited resolution of the data, local structural similarity restraints (LSSR; Smart et al 2012) were imposed during refinement. The target structures used for LSSR were the Tesidolumab Fab structure refined at 2.1 Å resolution and the free C5 structure derived from PDB entry 3CU7 (chain A), after Buster refinement using automated NCS restraints and the TLS groups originally defined by Fredslund et al (2008). This refinement step improved the Ramachandran statistics of the final model in comparison to the original PDB entry (79.5%, 18.6% and 1.1% of the residues in the core, allowed, and generously allowed regions of the Ramachandran plot, respectively, versus 74.9%, 23.0% and 1.5% for the original PDB entry). The final crystallographic model had R_{work} and R_{free} values of 23.3% and 29.3%, respectively, with a rmsd of 0.010 Å for bond lengths and 1.24° for bond angles.

### Analysis of the Structures

Structural overlays were performed with the programs Coot (Emsley et al 2010) or PyMOL (Molecular Graphics System; DeLano Scientific: Palo Alto, Calif.). The quality of the final refined models was assessed with the programs Coot and PROCHECK v3.3 (Laskowski et al 1992). Residues of human C5 that become less accessible to solvent upon binding of the TESIDOLUMAB antibody were identified by the program AREAIMOL of the CCP4 program suite (Collaborative Computational Project, Number 4, 1994).

### Results:

### Overall Structure:

Human C5 comprises a grand total of 13 structural domains. The β-chain (residues 19 to 673 of prepro-C5, Uniprot entry P01031) is made of six α-macroglobulin-like domains (MG1-6) and one linker domain. The α-chain (residues 678 to 1676) comprises the C5a (anaphylatoxin) domain, two α-macroglobulin-like domains (MG7, MG8), one CUB (“Complement C1r/C1s, Uegf, Bmp1”) domain, the thioester-like TED/C5d domain, and the carboxy-terminal C345C domain. The α-chain also contributes to the MG6 domain and is covalently attached to the β-chain through a disulfide-bridge within this domain.

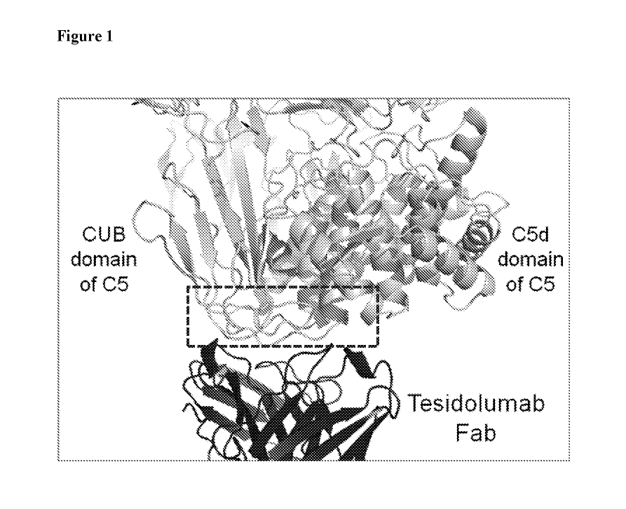

The tesidolumab Fab binds to the C5 α-chain, making contacts to both the CUB and TED/C5d domains (FIG. 1). The CUB domain possesses a β-sandwich fold, and the large, α-helical TED/C5d domain is inserted between strands β3 and β4 of the CUB domain. The peptide segment connecting the last α-helix of the TED/C5d domain to the β4 strand of the CUB domain runs through the antigen-combining site of the tesidolumab antibody and therefore constitutes one key component of the tesidolumab epitope.

### Tesidolumab Epitope on Human C5:

The tesidolumab Fab forms a 1:1 complex with human C5 and recognizes a discontinuous or “conformational” epitope on the target protein antigen, comprising 6 peptide segments in total (FIG. 1). The loop connecting the last α-helix of the TED/C5d domain (α12) to the β4 strand of the CUB domain (residues 1305-1310) plays a central role in the Tesidolumab epitope on C5. In addition three other peptide segments from the CUB domain contribute to the epitope: the β1′-β2 (residues 947-950), β5-β6 (residues 1327-1331) and β7-β8 (residues 1353-1354) loops. The TED/C5d domain also contribute two more structural elements to the epitope, the α2-α3 loop (residues 1029-1033) and the amino-terminal end of helix all (residues 1264-1265 and 1268).

C5 is a glycoprotein with four annotated N-linked glycosylation sites, at positions 741, 911, 1115 and 1630. Two of these glycosylation sites, at positions 741 and 911, have been observed by X-ray crystallography (Fredslund et al 2008). All four positions are remote from the epitope and therefore, the glycosylation state of the C5 antigen is not expected to affect Tesidolumab binding.

### The Connection Between the TED/C5d and CUB Domains of Human C5 Plays a Central Role in Tesidolumab Binding:

The connection between the TED/C5d and CUB domains runs approximately parallel to the VH/VL interface, along the central region of the antigen-combining site of tesidolumab. The amino-acid sequence of this peptide segment is 1305-Lys-Gln-Leu-Arg-Leu-Ser-1310. The side-chains of Lys1305 and Arg1308 are pointing towards the complementarity-determining regions (CDRs) of the antibody and are most likely contributing strong electrostatic interactions. Arg1308, in particular, is dipping into the central cavity of the antigen-combining site, lined by the L-CDR1, L-CDR3 and H-CDR3 hypervariable loops of the antibody. Therefore, the structure strongly suggests that Arg1308 plays a central role in tesidolumab recognition and binding of human C5 and that this residue is a hot spot of this protein-protein interface.

The C5 Polymorphism at Position 885 does not Affect the Tesidolumab Epitope:

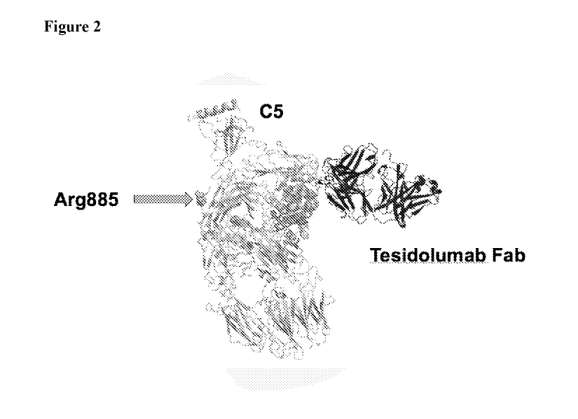

Eculizumab is a humanized anti-human C5 therapeutic antibody used for preventing complement-mediated hemolysis associated with PNH (Rother et al 2007). Two genetic variants of human C5 at position 885, the Arg885 to His and Arg885 to Cys variants, have been observed in patients showing a poor response to eculizumab treatment (Nishimura et al 2014). These C5 variants were functional but not blocked by eculizumab. Arg885 is found within the MG7 domain of C5. Inspection of the X-ray structure of the tesidolumab Fab complex shows that the location of Arg885 is remote from the tesidolumab epitope (FIG. 2). Therefore, C5 neutralization by tesidolumab is not affected by the Arg885 polymorphism observed in eculizumab non-responders.

### Example 2: MAC Formation C5 Demonstrates that Tesodulumab and not Eculizumab Inhibits Mutant C5

Tesidolumab and eculizumab were tested in a Wieslab assay using C5 depleted serum that was spiked with 7 μg/ml wt C5 (Arg885) or mutant C5 (His885).

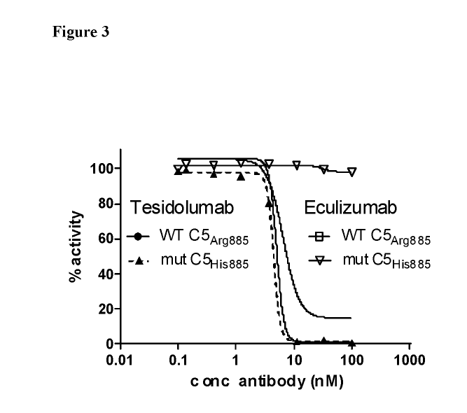

The results show that tesidolumab, but not eculizumab blocks membrane attack complex (MAC) formation in C5-depleted serum spiked with mutant C5. Both antibodies were equally potent in inhibiting MAC formation in serum spiked with wt C5. Tesidolumab was equally potent in serum spiked with normal or mutant C5. In contrast, eculizumab showed no activity in serum spiked with mutant C5 (FIG. 3).

### Example 3: Tesidolumab Shows Anti-Hemolytic Effects in C5 Variant and Non-Variant PNH

Open label, single-arm study to test tesidolumab (20 mg/kg i.v., two times a week) in C5 variant and non-variant PNH patients has been carried out.

### Methods:

To determine a pharmacodynamic response to tesidolumab, the capacity of the patients' serum to lyse antibody-sensitized chicken erythrocytes in a human serum-complement hemolytic assay can be measured (Hillmen et al., N Engl J Med 2004; 350:552-9). Less than 20% residual hemolysis is indicative of complete blockade of hemolysis in this assay system (Nishimura et al., New Engl J Med 2014; 370; 7).

### Results:

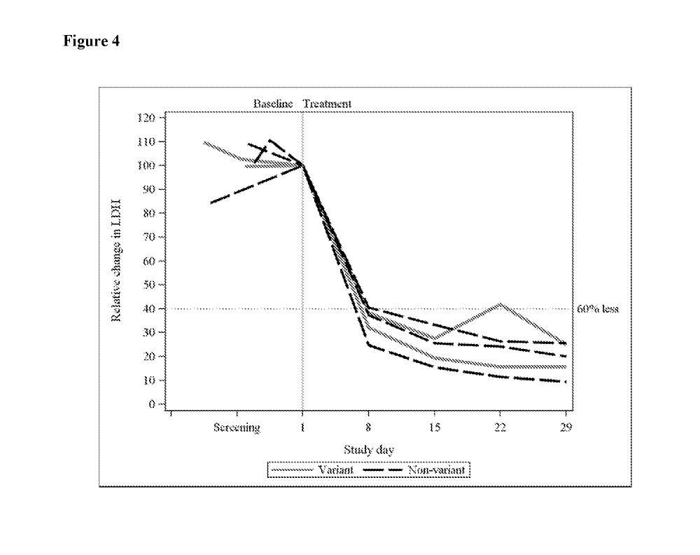

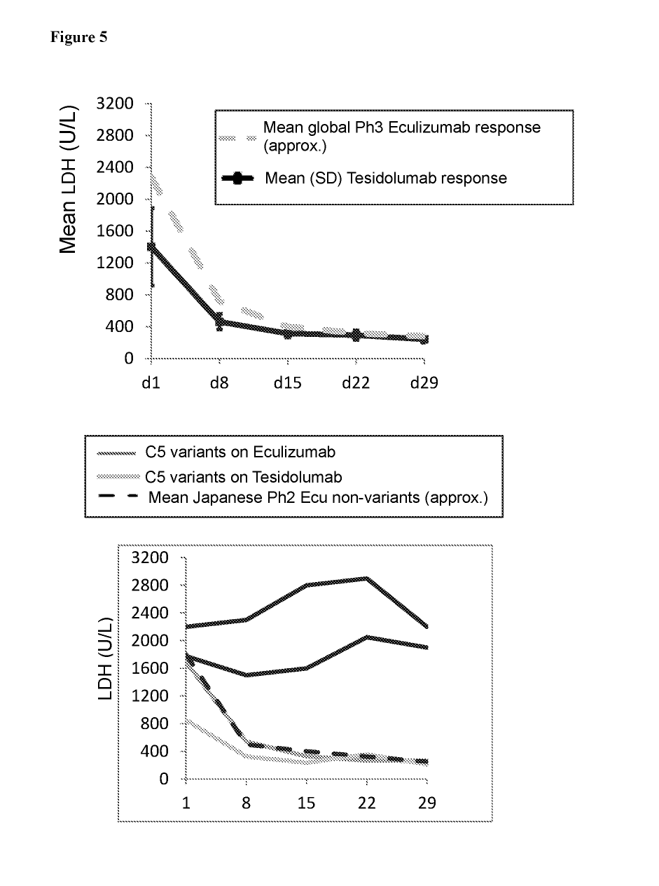

Analysis on the 5 patients (two C5 variants) was performed, after a mean treatment duration of 8.5 weeks. No major safety issues (no treatment discontinuation, no treatment related safety adverse event) were identified. An anti-hemolytic effect in PNH as evidenced by LDH reduction of 74-91% from baseline, was seen in both C5 variant and non-variant patients (FIGS. 4 and 5).

1. An anti-C5 antibody or antigen binding fragment thereof for use in the prophylaxis or treatment of a complement related disease or disorder in a patient who has a mutation or polymorphism within the MG7 domain of C5 protein or within an eculizumab epitope, e.g. a p.Arg885 polymorphism in complement C5 protein.

2. The anti-C5 antibody or antigen binding fragment thereof for use according to claim 1, wherein said anti-C5 antibody is capable of inhibiting complement activation in a patient who has a p.Arg885 polymorphism.

3. The anti-C5 antibody or antigen binding fragment thereof for use according to any one of the preceding claims, wherein said anti-C5 antibody is a human anti-C5 antibody.

4. The anti-C5 antibody or antigen binding fragment thereof for use according to claim 3, wherein said anti-C5 antibody is tesidolumab or an antigen binding fragment thereof.

5. The anti-C5 antibody or antigen binding fragment thereof for use according to any one of the preceding claims, wherein said patient has a p.Arg885His polymorphism.

6. The anti-C5 antibody or antigen binding fragment thereof for use according to any one of claims 1 to 4, wherein said patient has a p.Arg885Cys polymorphism.

7. The anti-C5 antibody or antigen binding fragment thereof for use according to any one of the preceding claims, wherein said C5 complement related disease is aHUS, PNH, bone marrow failure, aplastic anemia or thrombosis, e.g. PNH.

8. An anti-C5 antibody or antigen binding fragment for use as a medicament in a method comprising administering an effective amount of an anti-C5 antibody capable of inhibiting complement activation in a patient who has a mutation or polymorphism within the MG7 domain of the C5 protein or within the eculizumab epitope, e.g. a p.Arg885 polymorphism, to said patient.

9. An anti-C5 antibody or antigen binding fragment for use in a method of treating a complement related disease or disorder in a patient who has a mutation or polymorphism within the MG7 domain of the C5 protein or within the eculizumab epitope, e.g. a p.Arg885 polymorphism in complement C5 protein, wherein the method comprises administering an effective amount of an anti-C5 antibody to said patient, and wherein said anti-C5 antibody is capable of inhibiting complement activation in said patient.

10. The anti-C5 antibody or antigen binding fragment for use as a medicament in a method of claim 8 or 9, wherein said method comprises a step of determining from a biological sample obtained from the patient whether the C5 complement protein of the patient comprises either of a mutation or polymorphism within the MG7 domain of the C5 protein, within the eculizumab epitope, or a p.Arg885 polymorphism, wherein the biological sample is of tissue or fluid isolated from the patient.

11. An anti-C5 antibody or antigen binding fragment for use in a method of treating a complement related disease or disorder in a patient in need thereof, the method comprising:
a. taking a biological sample from the patient
b. screening for mutations or polymorphisms in the gene encoding C5 of said patient
c. determining whether the patient has either a mutation or polymorphism within the MG7 domain of the C5 protein, within the eculizumab epitope, or the p.Arg885 polymorphism in the C5 complement protein,
d. administering an effective amount of an anti-C5 antibody capable of inhibiting the complement activation in a patient who has at least a mutation or polymorphism as defined in step c),
wherein the biological sample is of tissue or fluid isolated from the patient.

12. The anti-C5 antibody or antigen binding fragment for use in the method of claims 9 to 11, wherein said complement related disease or disorder is a C5 complement related disease or disorder, e.g. PNH or aHUS.

13. The anti-C5 antibody or antigen binding fragment for use in the method of claims 8 to 12, wherein said anti-C5 antibody is tesidolumab or an antigen binding fragment thereof.

14. An anti-C5 antibody or antigen binding fragment for use in a method of treating PNH or aHUS, the method comprising:
a. determining from a biological sample obtained from a patient whether the patient has either a mutation or polymorphism within the MG7 domain of the C5 protein, within the eculizumab epitope, or a p.Arg885 polymorphism in the C5 complement protein, wherein the biological sample is of tissue or fluid isolated from the patient; and
b. administering an effective amount of tesidolumab or an antigen binding fragment thereof to said patient.

15. An anti-C5 antibody or antigen binding fragment for use in the prevention or treatment of a complement related disease or disorder, e.g. PNH or aHUS, in a patient in need thereof wherein the patient does not respond to eculizumab treatment.

16. The anti-C5 antibody or antigen binding fragment for use according to claim 14 or 15, wherein the patient has a p.Arg885 polymorphism in complement C5 protein.

17. Tesidolumab or an antigen binding fragment thereof for use in the prophylaxis or treatment of PNH or aHUS.

18. Use of an anti-C5 antibody or antigen binding fragment thereof for the manufacture of a medicament for the prophylaxis or treatment of a complement related disease or disorder, e.g. PNH or aHUS, in a patient who has a p.Arg885 polymorphism in complement C5 protein.

19. Use according to claim 18, wherein the anti-C5 antibody is tesidolumab or an antigen binding fragment thereof.
